(19) United States
(12) Patent Application Publication
(10) Pub. No.: US 2007/0031387 A1
(43) Pub. Date: Feb. 8, 2007

(54) Method for treating and/or preventing spinal cord injury

(75) Inventors: Chizuka Ide, Kyoto-shi (JP); Yoshihisa Suzuki, Kyoto-shi (JP); Masayoshi Ohta, Kyoto-shi (JP); Tomoyuki Yoshihara, Kyoto-shi (JP)

Correspondence Address: HAMRE, SCHUMANN, MUELLER & LARSON, P.C., P.O. BOX 2902, MINNEAPOLIS, MN, 55402-0902, US

(73) Assignee: KYOTO UNIVERSITY, Kyoto-shi (JP)

(21) Appl. No.: 11/500,230

(22) Filed: Aug. 7, 2006

Related U.S. Application Data
(60) Provisional application No. 60/706,610, filed on Aug. 8, 2005.

(51) Int. Cl. A61K 35/30

(52) U.S. Cl. 424/93.7

(57) ABSTRACT

A method of preventing or treating neurologic function deficit of vertebrates undergone spinal cord injury, comprising administering an effective amount of BM-MNCs to a vertebrate in need thereof through cerebrospinal fluid.

### CROSS REFERENCE

This application is based on provisional Application No. 60/706610 filed Aug. 8, 2005.

The patent or application file contains at least one drawing executed in color. Copies of this patent or patent application publication with color drawing(s) will be provided by the Office upon request and payment of the necessary fee.

### TECHNICAL FIELD

The present invention relates to a method for prevention and/or treatment of spinal cord injury, and more particularly, to the use of bone marrow-mononuclear cells in such a method.

### BACKGROUND ART

Spinal cord injury occurs due to traumatic injuries resulting from traffic accidents, athletic accidents, or falls and drops from heights, and to spinal cord compression, or the like. It also occurs due to other disorders, for example, when stroke is accompanied by pyramidal tract transection. Deficiency in neurologic function following spinal cord injury may progress in different ways; however, it is said that, in general, the neurologic function may be lost briefly due to concussion, for a longer period due to spinal cord compression caused by contusion or hemorrhage, and permanently due to lacerations or transection. In the case of human, totally severed or degenerated nerves in the cord cannot recover by itself and hence the damage is usually permanent. On the contrary, animal studies suggested that nerves damaged by contusion or transection could be regenerated.

Influences of spinal cord injury on neurologic functions vary depending on the site or severity thereof; however, injury in the upper cervical segments is often fatal and, even if a patient survives, the patient is so seriously affected that she/he is paralyzed in all four limbs and must live under assisted respiration. In the case where the injury is below the upper cervical segments, if the cord is completely damaged, flaccid paralysis of four limbs below the level of the injury, a loss of sensory and reflex functions, as well as an extinction of urinating or defecating functions may arise immediately. When the cord is incompletely damaged, the symptoms mentioned above may initially appear due to edema and softening of the central part of the spinal cord, then the patient may tend to recover from the injury in about 3-4 weeks; however, even in this case, there is a risk of aftereffects. Accordingly, an appropriate treatment at the right time is essential.

It is therefore important to treat as promptly as possible when the spinal cord is damaged, in order to promote recovery from or to prevent progress, of neurologic function deficit. It has been known that administration of high-dose steroid in the early stage after injury (generally, within 8 hours) is effective, but a high-dose steroid may cause side-effects.

Recently, treatment of spinal cord injury by grafting various cells (Schwann cells, olfactory ensheathing cells, neural stem cells, or embryonic stem cells, etc.) has been attempted. For example, a remedy for spinal cord injury comprising as an active ingredient glial cells such as type 1 astrocyte precursor cells, type 2 astrocyte precursor cells and 04 precursor cells was disclosed (WO2003/018041).

Further, a method of inducing mammal bone marrow stromal cells to differentiate into neuronal cells was disclosed, which method comprises contacting stromal cells with a compound capable of inducing the cells to differentiate into neurons (JP-A-2002-513545, WO99/56759).

Researchers including the present inventors have conducted histological and behavioral evaluations of rat models of spinal cord injury after transplanting peripheral nerve fragments, choroid plexus epithelial cells, neural stem cells or bone marrow stromal cells. Above all, bone marrow stromal cells transplanted by infusing into the cerebrospinal fluid brought certain results (Wu S., et al., J. Neurosci. Res., 2003 May 1;72 (3): 343-51; and Ohta M. et al., Exp. Neurol. 2004 June; 187(2): 266-78).

Among the above-mentioned conventional methods, those based on cell transplantation, except for the method using bone marrow stromal cells, are performed by transplanting neural stem cells or differentiated nerve cells into the injured sites. For example, a method using neuronal stem cells exerts therapeutic effects in the following manner: transplanted cells adhere to the injured site to survive, differentiate and proliferate to regenerate a nerve(s) having deficits. However, the local cell transplantation is highly invasive to patients and requires an advanced technique. Furthermore, it is not assured that all the transplanted cells (100%) survive and differentiate into nerve cells, and said method has a problem(s) such as low success rate. Even when stem cells are used after inducing to differentiate into nerve cells, this method of transplantation still has problems such as invasion to patients or necessity of advanced technology. It is not either assured that the transplanted cells survive and differentiate into nerve tissues. Moreover, these methods are hardly adapted to the emergency medical system which requires prompt care, considering the time necessary for the preparation of cells to be transplanted or for regeneration of nerve after transplantation.

On the other hand, in the therapeutic method which uses bone marrow stromal cells as disclosed by Wu et al. (ibid.) or Ohta et al. (ibid.), cells are not administered to the injured site but infused into spinal fluid. Therefore, said method is easy to operate and less invasive. However, the preparation of bone marrow stromal cells to be transplanted requires cultivation procedures even if the patient's own spinal cord is used, which comprise cultivation in a culture dish, separation of cells adhered to the dish, and optionally propagation of the adherent cells. Therefore, the method using bone marrow stromal cells is again hardly applicable to emergency medical system which requires prompt care.

Under the conditions, the development of a novel means capable of preventing or treating neurologic function deficits after spinal cord injury safely and properly even in an emergency situation has been strongly demanded.

### DISCLOSURE OF INVENTION

The purpose of the present invention is to provide an effective and highly safe method for prevention or treatment of neurologic function (neuronal) deficits after spinal cord injury.

The other purposes and effects of the present invention will be understood from the description and drawings.

It was unexpectedly found by the present inventors that bone marrow-mononuclear cells (BM-MNCs) have excellent preventive and therapeutic effects on the functional deficits of nervous system in rats with spinal cord injury, and established the present invention.

Thus, the present invention provides the followings:
1. A method for prevention or treatment of neurologic function deficits in a vertebrate with spinal cord injury, comprising a step of administering an effective amount of BM-MNCs to a vertebrate in need thereof through cerebrospinal fluid.
2. The method according 1, wherein the BM-MNCs are rich in cells positive for CD29, CD117, CD34, CD31 and/or CD11b/c.
3. The method according to 2, wherein the BM-MNCs contain CD34-positive cells at a percentage of 1.5% or higher.
4. A composition for prevention or treatment of neurologic function deficits in a vertebrate with spinal cord injury, which comprises BM-MNCs containing CD34-positive cells at a percentage of 1.5% or higher.

### BRIEF DESCRIPTION OF DRAWINGS

FIG. 1 shows the results of time course experiments wherein spinal cord injury models (Wister rats, male, 4-week-old) were transplanted with BM-MNCs by infusion into cerebrospinal fluid (CSF) through the fourth ventricle, and behavioral changes of the models was monitored, which BM-MNCs were harvested from the femoral bone marrow of an Wistar rat (male, 7-week-old) having been treated (A-group) or not treated (B-group) with 5-fluorouracil (5-FU). FIG. 1 also shows the results obtained in Control group and those obtained in the bone marrow stromal cell (BMSC)-transplanted group for comparison. In the figure, the control (o) represents the behavioral changes observed in the spinal cord injury models received the solvent (PBS) alone. BMSCS (Δ), MNCS(5FU+) (●), MNCS(5FU-1) (⋄) and MNCS(5FU-5) (♦) represent the behavioral changes observed in the spinal cord injury models transplanted with BMSCs, BM-MNCs harvested from a 5FU-treated rat, BM-MNCs (1×10⁷) harvested from a 5FU-untreated rat, and BM-MNCs (5×10⁶) harvested from a 5FU-untreated rat, respectively.

FIGS. 2A and 2B are micrographs showing the state of BM-MNCs in the lesion of spinal cord injury models (Wister rats male, 4-week-old) after 3 days from transplantation of BM-MNCs by infusion into cerebrospinal fluid (CSF) through the fourth ventricle, which BM-MNCs were harvested from the femoral bone marrow of an Wistar rat (male, 7-week-old) having been treated (A-group) or not treated (B-group) with 5-FU, respectively. In each figure, BM-MNCs are stained green with PKH67, and GFAP (glial fibrillary acidic protein), a marker protein of astrocyte, is red.

FIGS. 3A and 3B are micrographs showing the state of BM-MNCs in the lesion of spinal cord injury after 5 weeks from transplantation in A- and B-groups, respectively. In each figure, BM-MNCs are stained green with PKH67, and GFAP, a marker protein of astrocyte, is red.

FIG. 4A is a photograph of histological section of injured spinal cord after 5 weeks from transplantation in A-group stained with HE (haematoxylin and eosin). The control represents the result obtained in the untreated group. FIG. 4B is a photograph of histological section of injured spinal cord after 5 weeks from transplantation in B-group stained with HE.

FIG. 5 shows the cavity volume measured after 5 weeks from transplantation in Control group, BMSC-transplanted group, A-group, and B-group for comparison. The cavity volume is significantly small in A- and B-groups compared to that in Control group.

FIG. 6A is a photograph of preserved axons at the cavity-border of A-group shown in FIG. 4A. BM-MNCs (green) are found here and there among cells positive for β-tubulin type III (red, a neuronal cell marker). FIG. 6B is a photograph of preserved axons at the caudal side of the cavity-border of B-group shown in FIG. 4B, wherein β-tubulin type III (red) is a neuronal cell marker and glial fibrillary acidic protein (GFAP) (green) is a protein marker of astrocytes. FIG. 6C is a photograph of axons of Control group, which shows that neural components are not preserved at the caudal side of cavity. In the figure, β-tubulin type III (red) is a neuronal cell marker and glial fibrillary acidic protein (GFAP) (green) is a protein marker of astrocytes.

FIG. 7A is a photograph of vascular networks at the cavity-border of A-group shown in FIG. 4A, which shows von Wilebrand factor (red), BM-MNCs (green) and nuclei (blue) stained with TO-PRO-3 (Molecular Probes, Oregon, USA). FIG. 7B is a magnified view of a part of FIG. 7A, showing neovascularization. FIG. 7C is a photograph of neovascularization after 3 weeks from transplantation at the cavity-border of B-group shown in FIG. 4B. Blood vessels positive for von Wilebrand factor (red) are found around transplanted BM-MNCs (green). FIG. 7D is a photograph of neovascularization after 5 weeks from transplantation at the cavity-border of B-group and Control group. It is clear that there are greater neovascularization in even B-group compared to Control group.

FIG. 8 is a comparison of the number of vascular components within a defined area surrounding the cavity of A-group, B-group and Control group.

### BEST MODE FOR CARRYING OUT THE INVENTION

The present invention is described hereinafter in more detail

1. Treatment for Preventing or Treating Neurologic Function Deficits in a Vertebrate with Injured Spinal Cord

Treatment by the method of the present invention means preventing and/or treating deficits (also progress thereof of neurologic function (neurons) in a vertebrate with spinal cord injury. The kinds and conditions of neurologic function deficits caused by spinal cord injury are well known in the art. Disorders resulting from neurologic function deficits vary depending on the site and severity of injury, and may involve flaccid paralysis, loss of all sensation and reflex activity (including autonomic functions), partial motor and sensory loss, disorder of voluntary movement, and the like. The method of the present invention is useful in the prevention or treatment of any neurologic deficits which can be resulted from spinal cord injury.

Accordingly, the method of the present invention is applicable to lesions of partially or totally severed spinal cord. Further, the present method is applicable to injury at any sites without limitation, for example, brain, spinal cord segments proximal to brain such as medulla or cervical segment, and thoracic, lumbar, or sacral segments. There are no limitations regarding the symptoms of a patient to be treated either, and the present method is applicable to a patient suffering from moderate paralysis or severe symptoms such as quadriplegia, paraplegia, or respiratory paralysis.

The present method is preferably applied to exogenous spinal cord injuries resulting from trauma due to traffic accidents, athletic accidents, falls and drops from heights or due to compression, and the like. The present method is also useful for neurological disorders in the brain or spinal cord resulting from damages due to breakage of pyramidal tract at the time of stroke, or heart attack, or cardiovascular diseases (most are related to ischemia). Also, the present method is considered to be useful in the repair of damaged optic nerves.

The method of present invention is desirably applied to a patient within a short period of time, preferably within about 48 hours, more preferably within about 24 hours, and especially preferred within 8 hours, after spinal cord injury. However, the present method can be optionally used even after the above-mentioned period (for example, several days or weeks after injury), on condition that any effects can be expected.

The method of the present invention is contemplated treating vertebrates of any kinds including mammals, birds, fishes, specifically, human beings, domestic animals and pets such as cattle, horse, dog and cat, and domestic fowls.

2. Bone Marrow-Mononuclear Cells (BM-MNCs)

The “bone marrow-mononuclear cell (BM-MNC)” used in the method of the present invention refers to a mononuclear cell population obtained by removing granulocytes from bone marrow. Thus, BM-MNCs used in the method of the present invention also contain bone marrow stromal cells (BMSCs) and hematopoietic lineages (vascular endothelial progenitor cells or hematopoietic stem cells), and the like.

In the method of the present invention, BM-MNCs of any origin can be used as long as they are free from side effects to a patient. From the immunological view points, BM-MNCs from bone marrow of the patient with spinal cord injury or a blood relative thereof are preferred. However, other mononuclear cells such as those available from bone marrow bank or the like which are originated from bone marrow of a person other than blood relatives, those isolated from cord blood, and the like, can be used as long as they are immunologically acceptable. The preparation of BM-MNCs from such different origins above can be carried out by means of a commercially available kit, or by a method known in the art or taught in a literature (e.g., WO2001/066698, JP-A-2002-171965).

The so prepared BM-MNCs can be used as they are as suspension in, for example, an appropriate solvent (spinal fluid, physiological saline, distilled water, etc.). However, as described in the Examples below, the effects may be enhanced by increasing the percentage of hematopoietic lineages such as vascular endothelial progenitor cells and hematopoietic stem cells than usual. Accordingly, the present invention encompasses the use of BM-MNCs containing hematopoietic lineages at higher percentage.

Hematopoietic lineages (endothelial progenitor cells, hematopoietic stem cells, and the like) contained in BM-MNCs can be detected on the basis of cell markers (CD29, CD117, CD34, CD31 and CD11b/c). Examples of a method for increasing the percentage of hematopoietic lineages in the BM-MNCs include a method comprising administering an animal a substance capable of elevating the ratio of hematopoietic lineages such as 5FU, G-CSF, or the like, and harvesting bone marrow.

BM-MNCs used in the present method comprise hematopoietic lineages at a ratio normally found in mononuclear cells obtainable by the method described herein. In the case of human, the so obtained mononuclear cells generally contain hematopoietic lineages ranging from about 1.5 to 3%, which cells can be used in the present method. The percentage of hematopoietic lineages however can be higher or lower than the above-mentioned range, as long as it meets the purpose of the present invention. More specifically, CD34-positive cells among hematopoietic lineages are contained in the total BM-MNCs at 1.5% or higher, preferably at 10% or higher.

3. Administration of BM-MNCs

(1) The present method is preferably applied as early as possible after injury, more preferably within 48 hours, still more preferably within 24 hours, and further more preferably within 8 hours, after spinal cord is damaged, so that more excellent preventive and/or therapeutic effects on the neurological function deficits can be achieved. However, as a skilled person in the art can understand, the timing of treatment or the like is generally determined by a physician, and a patient can be treated at later stage depending on her/his conditions.

BM-MNCs are generally used for transplantation as a composition in the form of suspension in spinal fluid, physiological saline, distilled water, or the like. BM-MNCs can also be formulated into a composition suitable for administration as a suspension in physiological saline or an appropriate buffer such as PBS in such a manner that said composition contains hematopoietic lineages at a higher percentage, if needed. Alternatively, BM-MNCs can be cryopreserved in physiological saline, and reconstituted by suspending into a solvent above before use. The method of isolation and preservation of BM-MNCs, and preparation of a composition are known to a skilled person in the art pertaining to cell transplantation. Such a composition of BM-MNCs is useful when it is difficult to obtain BM-MNCs from the patient.

The composition of the present invention may contain any pharmaceutically acceptable additive so long as it does not affect adversely the action of BM-MNCs. There is no limitation to the cell density in the composition; however, it is generally from about 10³ to about 10⁹ cells/μl, preferably from about 10⁴ to about 10⁸ cells/μl, more preferably, from about 10⁴ to about 10⁷ cells/μl.

Thus, as another embodiment, the present invention provides a composition comprising BM-MNCs for preventing or treating neurologic function deficits in a vertebrate with spinal cord injury.

BM-MNCs used in such a composition preferably contain hematopoietic lineages at a higher percentage than usual BM-MNCs do.

(2) The present therapeutic method is characterized in that an effective amount of BM-MNCs are directly infused into cerebrospinal fluid of a patient.

The method of administrating BM-MNCs to a patient via cerebrospinal fluid is not limited to any method as long as cells can be infused safely and certainly. For example, cell suspension can be infused through a site suitable for isolating cerebrospinal fluid (e.g., the fourth ventricle, in case of human, subarachnoid of lumbar region, dilated cerebral ventricle due to hydrocephalia, or the like).

The number of cells to be infused varies depending on the age, sexuality, body weight of the patient and location or severity of injury, and the like; however, about 10⁶-10⁸ cells in total is necessary for a patient. It is preferred to administer a greater number of cells though, an appropriate number should be decided by the attending physician, and more or lesser number of cells are also usable.

(3) When a patient is treated with BM-MNCs harvested from her- or himself, immunologic rejection does not occur. When BM-MNCs originated in bone marrow of others or those prepared using umbilical fluid or the like are used, an immunosuppressant may be preliminarily administered to the patient. An immunosuppressant used for this purpose can be selected from those generally used in the bone marrow- or organ-transplantation. Examples include cyclosporin, tacrolimus hydrate (FK506), cyclophosamide, azathioprine, mizoribine and methotrexate. The dosage of immunosuppressant can be determined appropriately considering the kinds of the drug, origin of BM-MNCs to be administered, tolerance of the patient, and the like.

4. Preventive or Therapeutic Effects of BM-MNCs on Spinal Cord Injury

As shown in the working Example below, when rats undergone crush injury in spinal cord were treated by the present method, prevention of spinal cord injury and repair of contusion lesion were more evident in the treated group compared to the control group from the viewpoints of histological findings and behavioral evaluations based on BBB scores. For example, the following observations can be listed as characteristic features of the method of the present invention which comprises administering BM-MNCs into cerebrospinal fluid.

1) BM-MNCs migrate to the lesion of crush injury in the acute phase after administration, and then gradually decrease in number, and become hardly found after several weeks.

2) Differentiation of BM-MNCs into neuronal cells (neurons, glial cells) or vascular endothelial cells is not observed.

3) Significant reduction of the volume of cavity formed by crush is observed.

4) Significant improvement of BBB score is observed.

5) A significantly greater number of new vessels are generated around the cavity.

6) More neural components are preserved in the caudal region than in the lesion of crush injury.

As mentioned above, BM-MNCs administered to spinal fluid by the present method (therapy for spinal cord injury by transplantation of BM-MNCs) are broadly distributed from brain to spinal cord via cerebrospinal fluid, and partly reach the lesion (injured site). BM-MNCs distributed in brain and spinal were revealed to have effects (actions) such as promotion of neovascularization, protection of cells and acceleration of nerve regeneration, and to be effective in the treatment or prevention of neurologic function deficits at the site of injury.

The above-mentioned effects of BM-MNCs are thought to be attributable to factors released from constituent cells. For example, factors from stromal cells include nerve growth factors which prevent degeneration, inhibit apoptosis and/or promote recovery from degeneration of spinal nerve, and factors capable of promoting differentiation of endogenous neural stem cells; and factors from hematopoietic lineages (endothelial progenitor cells or CD34-positive hematopoietic stem cells) include unknown secretor factors which enhance neovascularization (hyperplasia).

In the case of BM-MNCs from bone marrow of a 5FU-treated rat, the percentage of hematopoietic lineages is increased and is about 6-times as high as that of untreated group. The fact that BM-MNCs rich in hematopoietic lineages have higher preventive and/or therapeutic effects on the neurologic function deficits than BM-MNCs from a 5FU-untreated rat indicates that factors from hematopoietic lineages are important.

On the other hand, the fact that BM-MNCs from a 5FU-untreated rat exert effects equivalent to the effects obtained at the time of administration of known BMSCs, while showing a significant difference from Control group, clearly indicates that the therapeutical effects of the present invention can be achieved without administration of 5FU. Thus, in this manner wherein preliminary administration of 5FU is unnecessary, the present method can provide a effective therapy for treating a patient at early stage, that is, as soon as the patient is carried in and diagnosed.

The method of the present invention can be performed by administering BM-MNCs into cerebrospinal fluid, and therefore it is less invasive and safer, and imposes a patient lower burden than a method which is performed by transplanting cells to the site of injury (injured lesion). Further, BM-MNCs being prepared without cultivation procedures, the treatment of a patient can be facilitated. In particular, when a patient's own BM-MNCs are used, the present method allows to treat the patient promptly and safely with a simple equipment, yet achieves high effects. Thus, the present invention can greatly contribute to the prevention and treatment of spinal cord injury accompanied to accidents or the like which is a matter of urgency. When BM-MNCs previously prepared for transplantation are used, for example, a patient from whom BM-MNCs are hardly harvested can be treated, and further, the preventive or therapeutic effects on spinal cord injury can be improved by increasing the percentage of hematopoietic lineages, if desired.

Furthermore, BM-MNCs can be harvested easily more than once, which makes it possible to treat a patient plural times continuously, when needed, whereby functional loss due to secondary enlargement of cavity can be prevented.

### EXAMPLE 1

### Effects of BM-MNCs on Spinal Cord Injury

In the experiments, Wistar rats (male, 4-week-old, 6 animals/group) were used under peritoneal anesthesia with pentobarbital.

1. Construction of Spinal Cord Injury Models

The spinal cord of Wister rats (male, 4-week-old) was exposed by elevating the arcus vertebrae in a valve-like shape at the level of thoracic vertebrae (T8-9). According to the world-wide standard method for preparing spinal cord injury models, contusion injury was made by dropping a weight (10 g, 2 mm in diameter) from 12.5 mm in height by means of a spinal cord injuring device “New York University Weight Drop Device”. The dura was not injured. After restoring the arcus vertebrae, the wound was sealed.

2. Bone Marrow Mononuclear Cell (BM-MNC)

(1) Preparation of BM-MNCs

BM-MNCs were harvested from both femora of Wistar rat (male, 7-week-old) in a test tube containing Lymphoprep™ and supernatant containing BM-MNCs was obtained by differential centrifugation (density gradient using Lymphoprep™ density solution (density: 1.077); Nycomed Pharma, Norway). Cells were then labeled with PKH67 (MINI-67; Sigma) according to the accompanying instructions. Behavior of PKH67-stained cells can be traced on the basis of green fluorescence.

Bone Marrow-Mononuclear Cell A (BM-MNC-A)

To a rat was administered once 5-fluorourasil (5FU) (150 mg/kg), and three days later, BM-MNCs were harvested from femora and labeled in the same manner as above.

Bone Marrow-Mononuclear Cell B (BM-MNC-B)

BM-MNCs were harvested from femora of a rat without preliminary administration of 5-FU and labeled in the same manner as above.

(2) Analysis of BM-MNCs

The constituent cells of the above-mentioned BM-MNC-A and BM-MNC-B were analyzed by cell sorter (FACS, FACScalibur and CellQuest software, Becton Dickenson and Co. USA) to analyze the cells constituting the BM-MNC-A and BM-MNC-B. Further, the differentially harvested BM-MNCs were cultured in a culture dish (10 cm in diameter) containing α-MEM (Sigma, St Louis, Mo.) to examine the presence or absence of cells adhering to the dish.

The results are shown in Table 1.

TABLE 1
Constituent of BM-MNCs

| | A | B | |
|---|---|---|---|
| CD90 (Thy1) | 43.1% | 67.9% | Thy1 |
| CD45 | 38.2% | 79.8% | Leukocyte common antigen |
| CD29 | 73.5% | — | B1 integrin |
| CD117 | 11.3% | 1.7% | c-kit |
| CD34 | 10.3% | 1.9% | hematopoietic stem cell marker |
| CD31 | 23.5% | — | endothelial cell marker |
| CD11b/c | 18.5% | — | macrophage |

%: percentage (%) to the total cells used for transplantation
—: not detected
Thy1, CD29: stromal cell marker
Thy1, c-kit: endothelial cell marker

As is clear from Table 1, the percentage of cells with hematopoietic marker was increased in 5FU-treated A-group (about 10%) by about 5 to 6 times compared to 5FU-untreated B-group (about 2%).

In addition, both of A- and B-groups contained adherent cells (assumed to be BMSCs) at a percentage of 10% or below in the cells harvested as BM-MNCs. Immunohistological analysis of the adherent cells revealed that they were positive for stromal cell markers [CD29 (B 1-integrin), CD90 (Thy-1)], but negative for hematopoietic cell marker (CD34, CD45), blood vascular marker (CD31), or macrophage markers (CD11b/c). Since adherent cells were negative for CD34, they were supposed to be cells other than hematopoietic progenitors. Nonadherent cells were of hematopoietic lineages, and hematopoietic markers such as CD34 were increased by 5- to 6-fold in A-group.

3. Administration of BM-MNCs

The labeled BM-MNCs (A-group) prepared in 2 above were suspended in PBS at 10⁵ cells/μl, and administered to a spinal cord injury model prepared in 1 above about one hour after injury. Specifically, a spinal cord injury model was drilled a hole in the skull and fixed to a stereotaxic instrument. The labeled BM-MNCs (50 μl, 5×10⁶ cells) were injected into the rat fourth ventricle through the hole with a needle attached to a syringe at the depth level of 6.25 mm over 5 minutes. A rat of Control group was administered with PBS alone (50 μl).

Rats were treated in the same manner as above using suspension (50 μl) containing BM-MNCs (B-group) at a cell density of 1'10⁵ cells/μl or 2×10⁵ cells/μl in PBS (number of transplanted cells; 5×10⁶ or 1×10⁷). BMSCs were harvested, cultured and suspended in PBS at 1×10⁵ cells/μl, and used to treat a rat in the same manner as above (number of transplanted cells; 5×10⁶).

4. Effects of Transplanted BM-MNCs on Behavioral Function

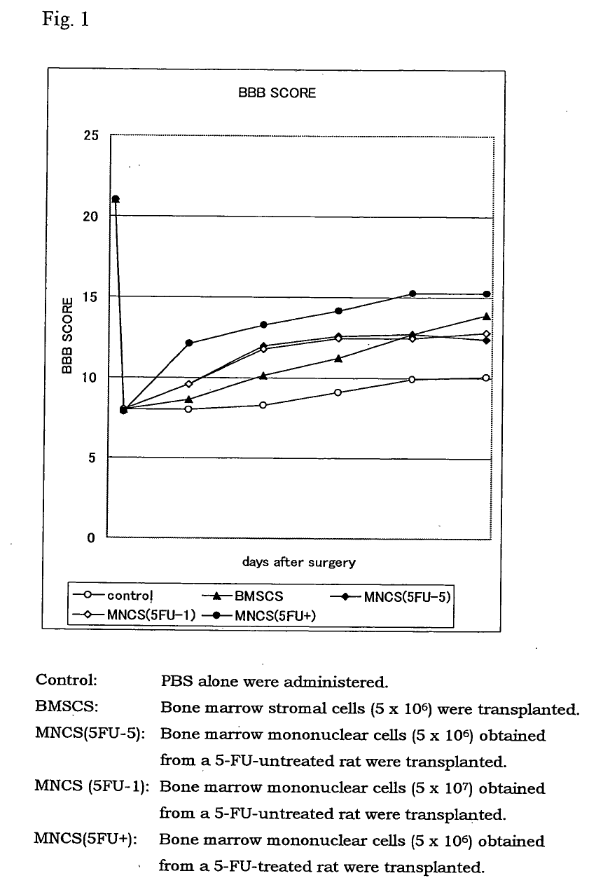

Behavioral function of rats was evaluated on the basis of BBB score before spinal cord injury, and one day, 2, 3, 4 and 5 weeks after spinal cord injury. The BBB score method is a known test system for the evaluation of hindlimb locomotion function (BBB score), wherein the measurement of function is classified into 21 grades from score 0 (complete paralysis) to 21 (normal). FIG. 1 shows the time-dependent changes of BBB scores of rats in A- and B-groups until 5 weeks after transplantation. In the figure, “BMSCS” refers to the behavioral function of animals transplanted with BMSCs; “MNCS(5FU+)” to the behavioral function of animals transplanted with BM-MNCs from rats treated with 5-FU; “MNCS(5FU-1)” to the behavioral function of animals transplanted with 1×10⁷ BM-MNCs from rats not-treated with 5-FU; and “MNCS(5FU-5)” to the behavioral function of animals transplanted with 5×10⁶ BM-MNCs from rats not-treated with 5-FU,. The behavioral function of rats was significantly improved 1, 2, 3, 4 and 5 weeks after injury in both of A- and B-groups compared to Control group (rats received PBS). The BBB scores after five weeks from day 1 when rats were injured and treated by transplantation are 15.3±3.60 in A-group (5FU+ group), 12.77±23.00 in B-group (MNCS(1)5FU-1), 12.4±2.60 in B-group (MNCS (5)5FU-5), and 10.1±2.10 in Control group. The present inventors conducted the same experiments using BMSCs. As a result, BBB score on week 5 was 13.875±3.0 (Ohta, M. et al., 2004, Exp. Neurol. 187, 266-278). It is evident that the behavioral function can be improved in A- and B-groups compared to Control group. Further, the both of MNCS(1) and MNCS(5) groups showed almost the same effect which is equivalent to that of known BMSCs. Thus, BM-MNCs harvested without administration of 5FU, when a certain number of cells are present, can exert an effect. However, it is desirable that BM-MNCs contain hematopoietic stem cells at a high percentage. FIG. 1 shows that the present method using BM-MNCs has an effect of improving behavioral function after spinal cord injury to a similar or greater extent than the method using BMSCs.

5. Histological Analysis

(1) Method

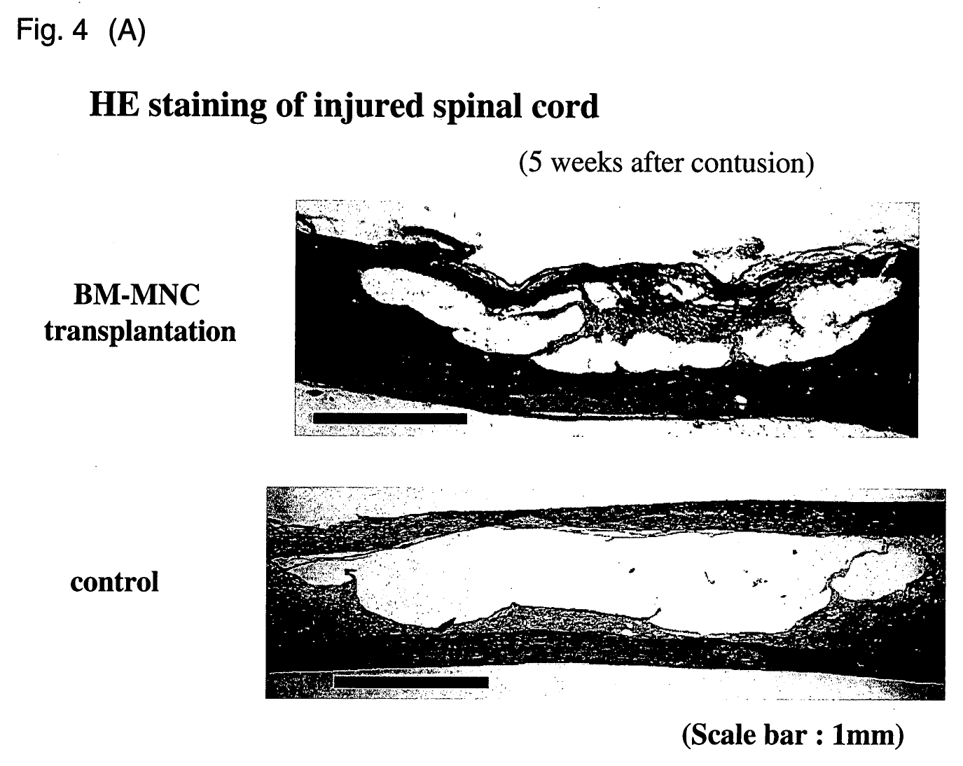

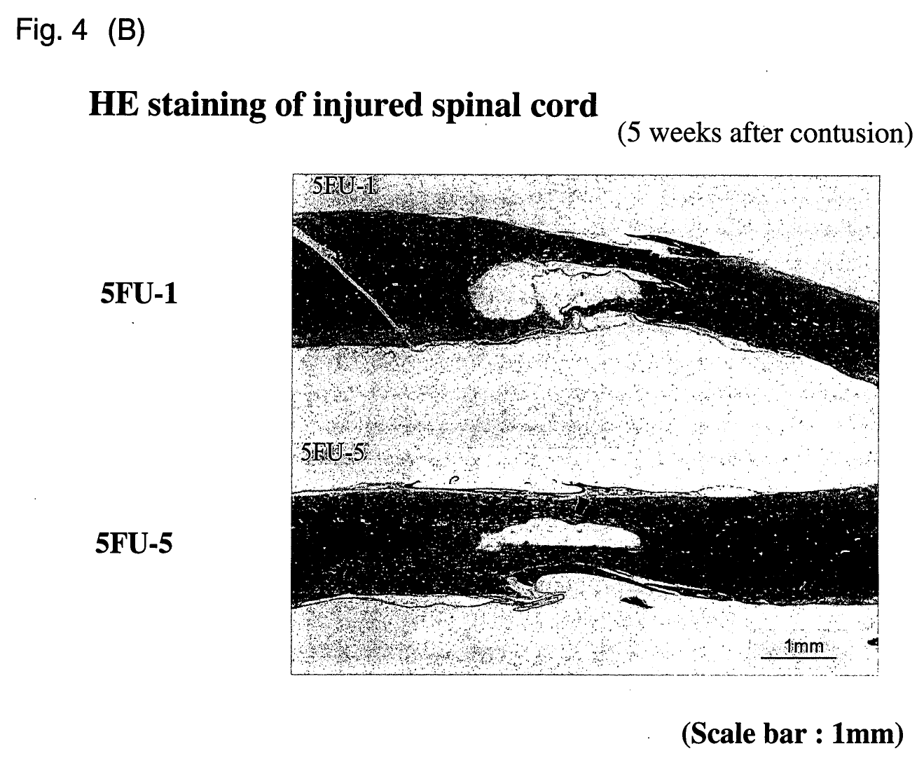

The therapeutic effects of transplantation on spinal cord injury in rats of A-group were examined on the basis of changes in transplanted cells, neovascularization (angiogenesis) at the injured site of spinal cord, and the volume of cavity. The structure and the conditions of phagocytes at the injured site were monitored after 1, 3 and 4 days and 1, 2, 3, 4 and 5 weeks from transplantation (FIGS. 4, 6, 7, 8). Further, rats of which behavioral function was observed and examined for 5 weeks were fixed after observation and the cavity volume was measured and evaluated. In addition, histological analysis was conducted by HE-staining of histological sections.

Specifically, spinal cord was removed from the injured site of an animal, frozen, cut into sections (10 μm), and stained with HE in every 10 sections. The rate of volume integration was calculated out on the basis of respective areas.

(2) Results

Migration of Transplanted BM-MNCs

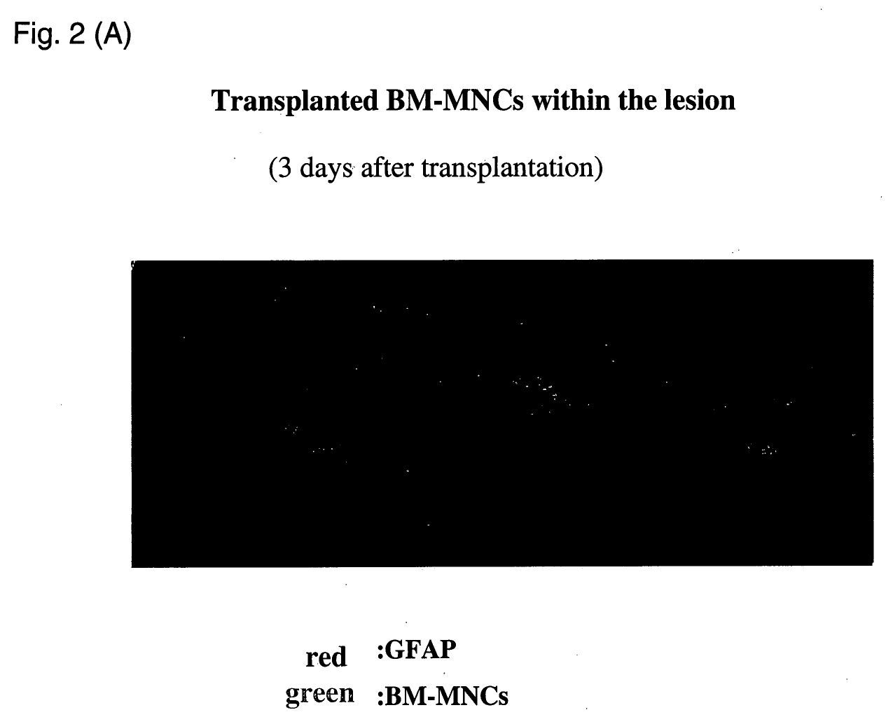

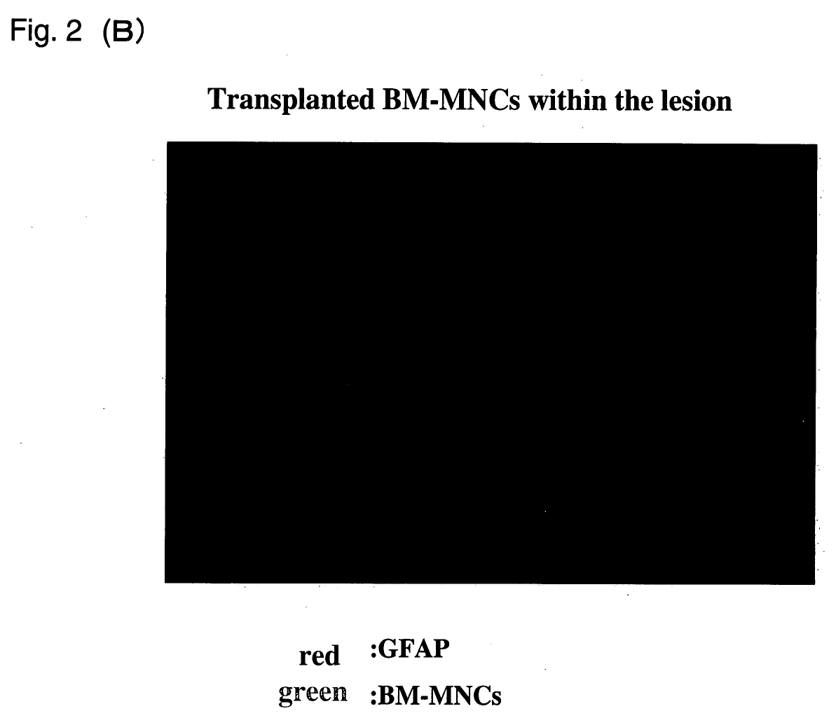

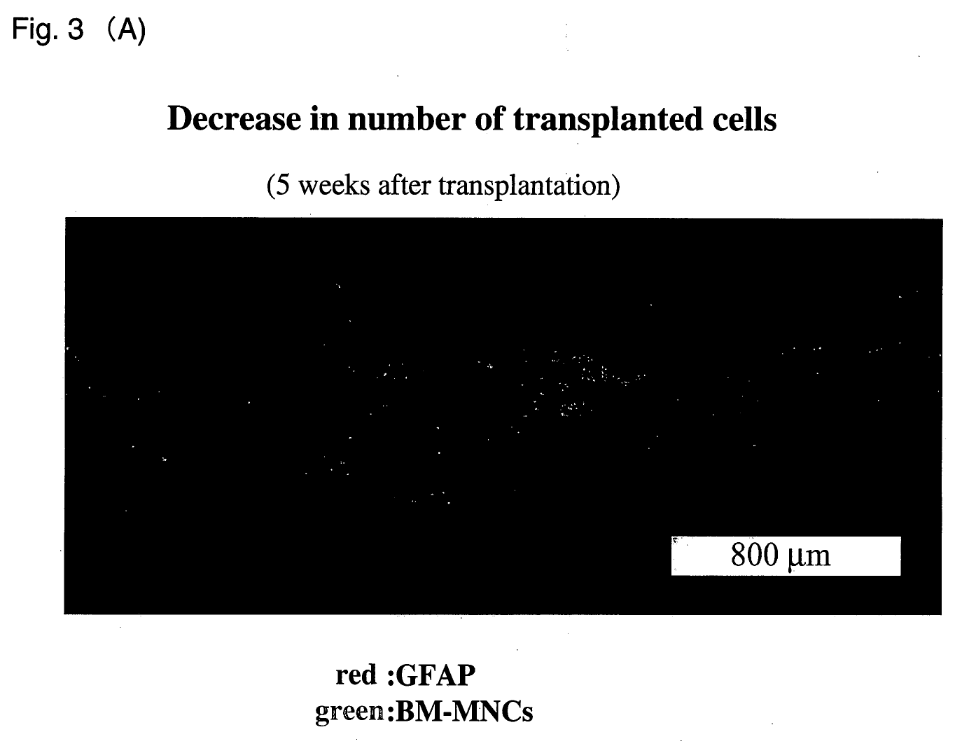

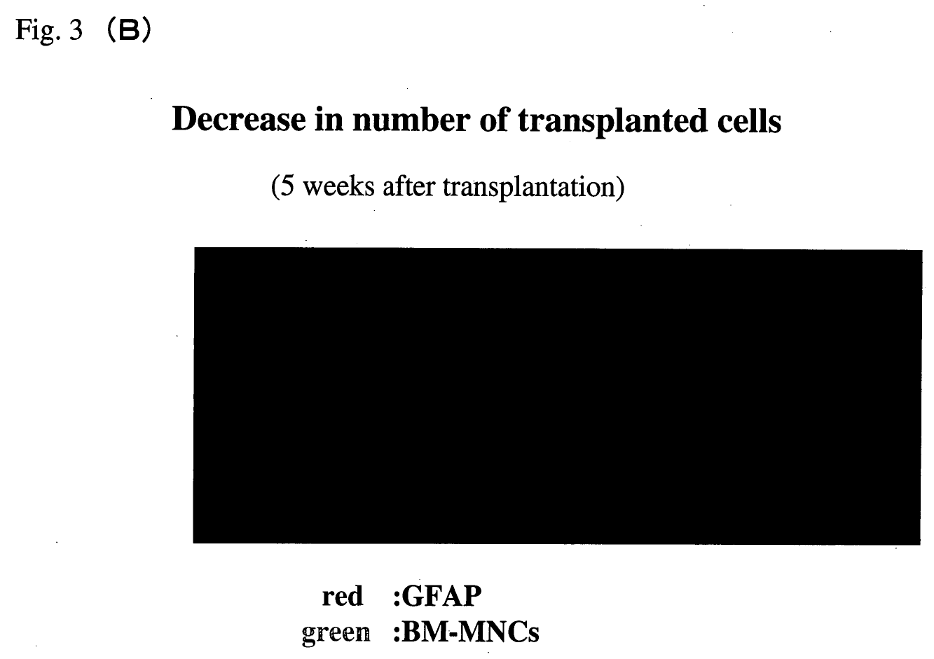

FIGS. 2A and 2B are micrographs showing the state of BM-MNCs within the lesion after 3 days from transplantation. FIGS. 2A and 2B show that relatively many transplanted cells entered the tissue of injured spinal cord on day 3 in both A- and B-groups. However, BM-MNCs in the lesion decreased in number gradually and almost disappeared after 5 weeks (FIGS. 3A, B). In the Figure, “BM-MNC” represents bone-marrow mononuclear cells and are colored green by PKH67 staining. GFAP (glial fibrillary acidic protein) is a marker protein of astrocytes and colored red.

Differentiation of Transplanted BM-MNCs

As shown in FIGS. 3A and B, there were no evidences suggesting that transplanted cells differentiated into neurons or glial cells in A-group. As far as the inventors examined, cells did not differentiated into vascular endothelial cells either (transplanted cells were not found and thought to have disappeared). In B-group, similar results to the above were obtained.

Haematoxylin and Eosin (HE) Staining

Histological sections (10 μm) were prepared from rat injured spinal cord, stained with HE in a conventional manner, and subjected to histological examination. The results are shown in FIGS. 4A and B. It is clear from FIGS. 4A and B that cavity volume is decreased in the tissue undergone transplantation of BM-MNCs.

FIG. 4A is a photograph of HE-stained histological section of the same lesion (5 weeks from injury) as that shown in FIG. 2. In Control group, PBS was infused. In the treated group (A-group), broader area is stained red with eosin (cytoplasm, connective tissue, erythrocytes, etc.) than Control group, indicating that damaged tissue was more efficiently regenerated (repaired). FIG. 4B is a photograph of HE-stained histological section of injured spinal cord in B-group after 5 weeks from transplantation. In B-group, similar results to the above were obtained.

Change in Cavity Volume

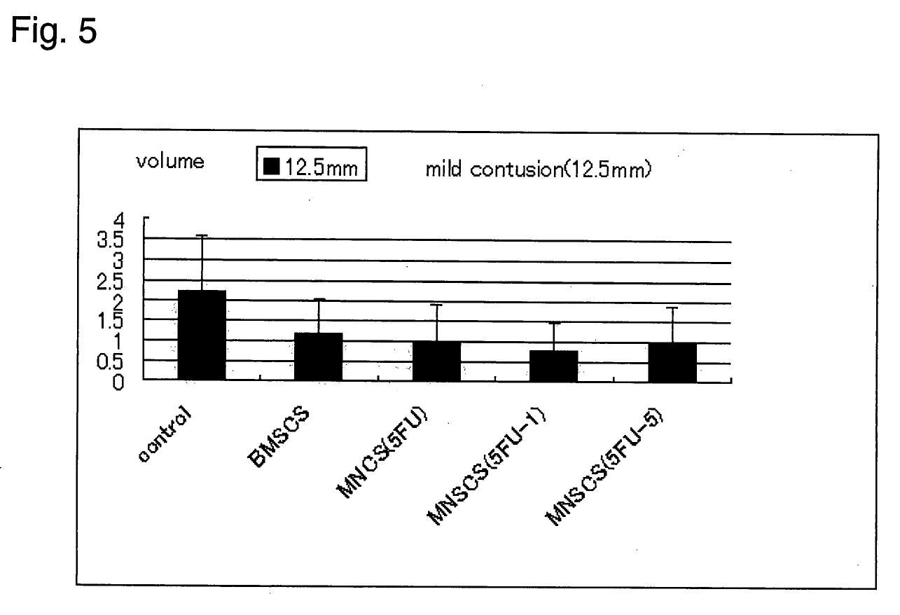

The change in the volume of cavity formed after spinal cord injury was monitored. The results are shown in FIG. 5. As is clear from the figure, the cavity volume is decreased significantly in the BM-MNC-transplanted group compared to Control group. That is, the cavity areas after five weeks from the day of injury (day 1) are 1.01±0.389 mm³ in A-group, 0.78±0.7 mm³ in MNCS(1)5FU-1 group of B-group, 0.96±0.9 mm3 in MNCS(5)5FU-group of B-group, and 2.22±0.199 mm³ in Control group.

When the present inventors conducted experiments in the same manner using BMSCs, the cavity volume after five weeks was 1.18 mm³ (Ohta M, et al., Exp Neurol. 2004, June;187(2): 266-78). The above results show that the present method exerts higher cavity reduction effect than the method using BMSCs.

Formation of Axon and Neovascularization

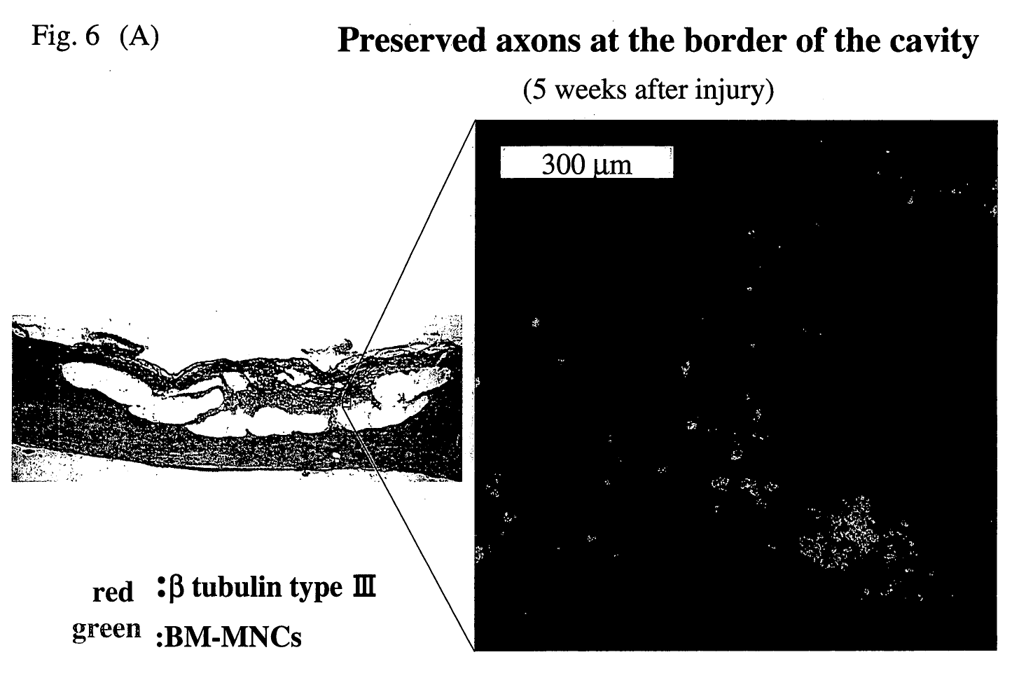

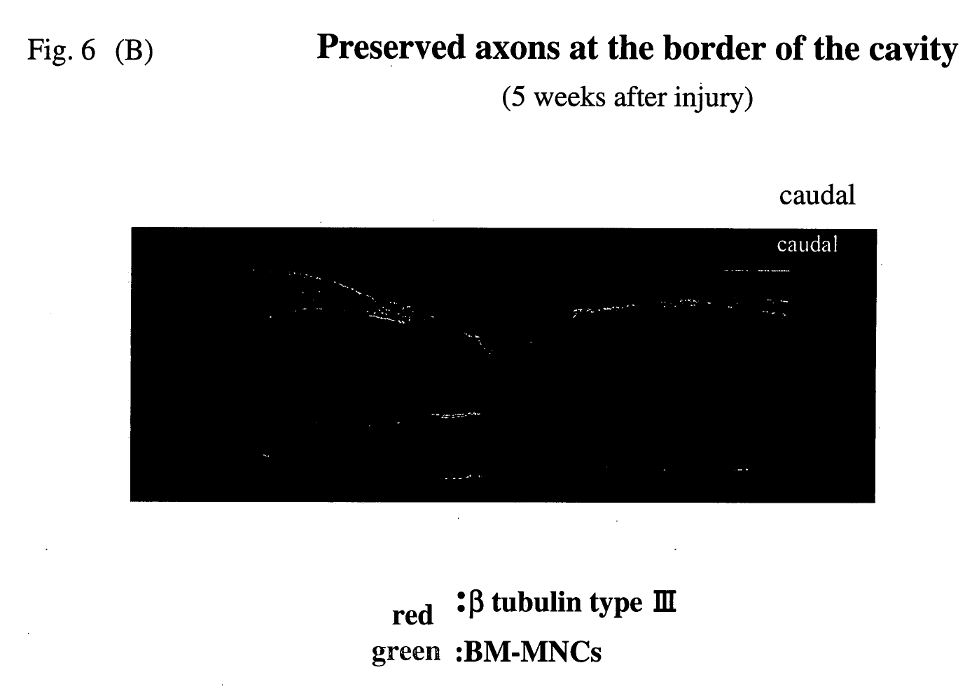

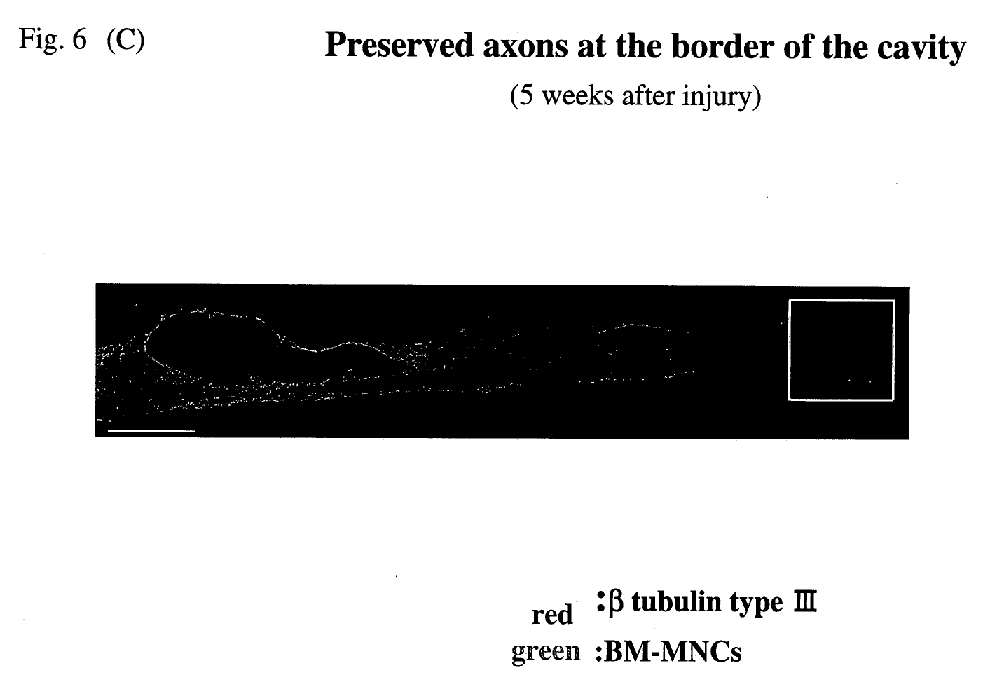

FIG. 6A is a micrograph showing axons preserved at the border of the cavity of A-group shown in FIG. 4A. BM-MNCs (green) are found here and there among β-tubulin type III (red) which is a neuronal cell marker. FIG. 6B is a micrograph showing that neural components are preserved at the caudal site of border of the cavity in B-group shown in FIG. 4B. FIG. 6C shows that neural components are not preserved at the caudal site of cavity in Control group.

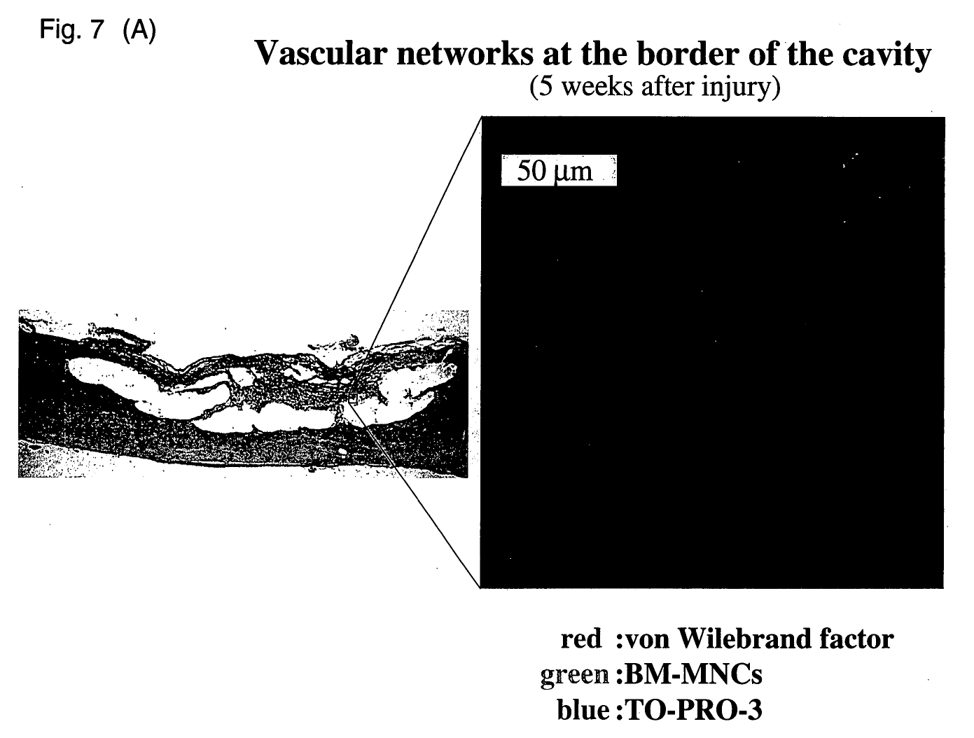

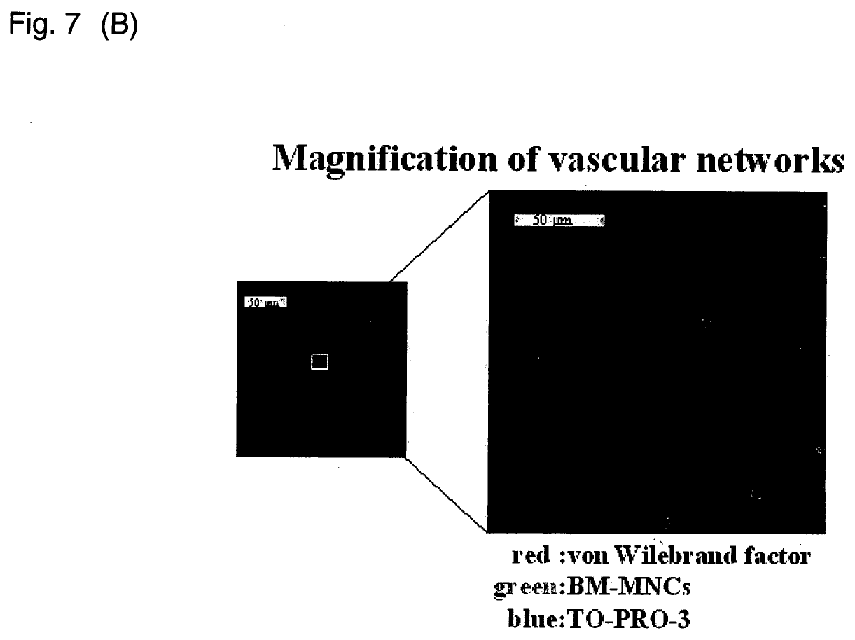

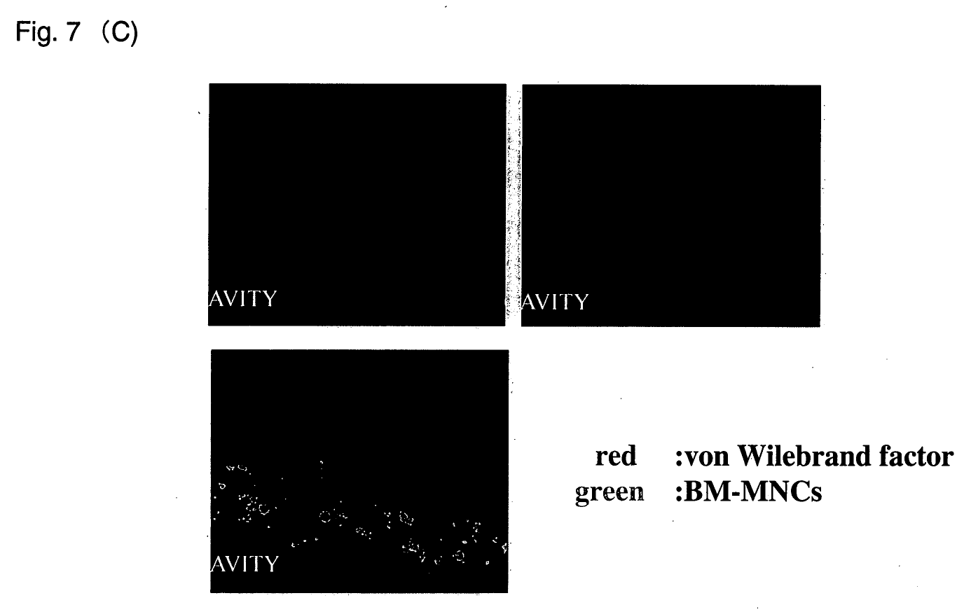

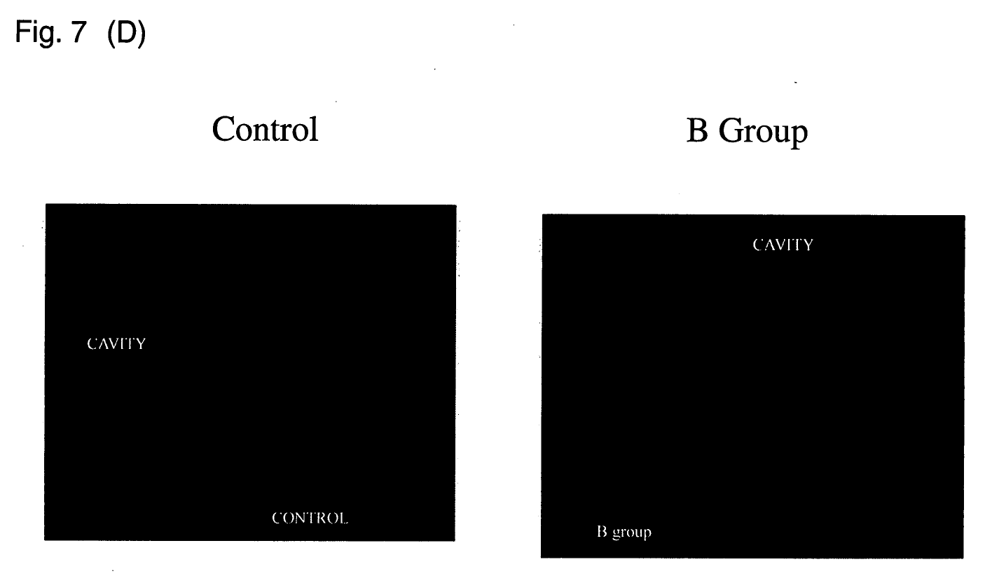

FIG. 7A is a photograph of vascular networks at the border of the cavity of A-group shown in FIG. 4A, wherein von Wilebrand factor (red), BM-MNCs (green) and nuclei (blue) stained with TO-PRO-3 (Molecular Probes, Oreg., USA) are shown.

FIG. 7B is a micrograph of magnified view of a part of FIG. 7A. FIG. 7B shows that vessels are constructed around neural tissue which was probably preserved as a result of administration of mononuclear cells.

FIG. 7C shows neovascularization after 3 weeks from transplantation at the border of the cavity of B-group shown in FIG. 4B. Blood vessels positive for von Wilebrand factor (red) are found around transplanted BM-MNCs (green).

FIG. 7D shows neovascularization after 5 weeks from transplantation at the border of the cavity of B-group and Control group. It can be seen that neovascularization is more abundant also in B-group compared to Control group.

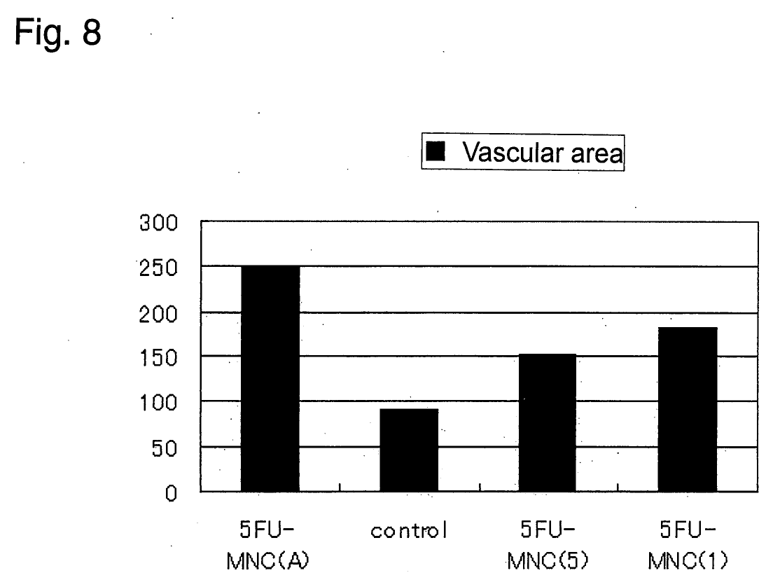

FIG. 8 shows the number of vascular components within a defined area surrounding the cavity of A-group, B-group and Control group for comparison. The vascular network density was significantly higher in A- and B-groups compared to Control group.

The results above show that administration of BM-MNCs of A-group promotes neovascularization.

### DISCUSSION AND INDUSTRIAL APPLICABILITY

The behavioral and histological analyses revealed that neurologic function deficits in rats with spinal cord injury was significantly improved when treated by the method of the present invention. The experimental results suggest that BM-MNCs infused into spinal fluid migrate through the spinal fluid, adhere to spinal cord, and a portion reaches the injured lesion and enters the spinal cord cavity.

Specifically, it was revealed that BM-MNCs of A- and B-groups contain vascular endothelial progenitor cells and BMSCs at a low percentage though, that the preventive or therapeutic effects on spinal cord injury of BM-MNCs of A- and B-groups are equivalent to or higher than the effects obtained when BMSCs are transplanted (Wu S., et al., J. Neurosci. Res. 2003 May 1;72(3): 343-51; and Ohta M. et al., Exp. Neurol. 2004 June; 187(2): 266-78), and that BM-MNCs of A-group which contain cells (about 10%) positive for hematopoietic stem cell marker (CD34, CD117) about 5-6 times as many as those of B-group (1.9%), exert significantly higher preventive and therapeutic effects on spinal cord injury than the effects obtained by the known method where BMSCs are transplanted. However, BM-MNCs of B-group, which contain hematopoietic lineages within the normal range, also exerted almost similar effects in the improvement of behavioral function and histological restoration to the effects obtained by the known method where BMSCs are transplanted. Further, notwithstanding that A-group contains BMSCs to be transplanted at a lower percentage (see, Table 1), A-group rich in hematopoietic lineages exerts higher therapeutic effects than the effects obtained by known method where BMSCs are transplanted, indicating that neovascularization and revascularization by secretory factors from vascular endothelial progenitor cells and/or hematopoietic stem cells, in addition to those from BMSCs, contribute to the expression of preventive and therapeutic effects on the spinal cord injury.

Because nerve is subject to ischemia, maintenance of blood flow is significant for the protection of nerve tissue and eventually for the nerve regeneration. The results obtained in B-group of the present invention suggest that BM-MNCs containing vascular endothelial progenitor cells and hematopoietic stem cells at the normal ratio found in those harvested and isolated under ordinary conditions also exhibit therapeutic effects on the spinal cord injury. Furthermore, it became clear from the results obtained in A-group of the present invention that, in the treatment of spinal cord injury, the prevention and regeneration of vessels are promoted and the effects of repairing injured nerve is enhanced when the content (percentage) of vascular endothelial progenitor cells or hematopoietic stem cells in the BM-MNCs are increased. It was suggested that more effective means of preventing or treating neurologic function deficits of injured spinal cord can be developed by increasing the percentage of vascular endothelial progenitor cells and/or hematopoietic stem cells in BM-MNCs artificially through the preliminary administration of G-CSF, or the like.

Thus, the present invention provides a method for preventing and/or treating neurologic function deficits after spinal cord injury safely and effectively, and whereby greatly contribute to the improvement of quality of medical care, especially emergency medical care.

What is claimed is:

1. A method for prevention or treatment of neurologic function deficits in a vertebrate with spinal cord injury, comprising a step of administering an effective amount of BM-MNCs to a vertebrate in need thereof through cerebrospinal fluid.

2. The method according 1, wherein the BM-MNCs are rich in cells positive for CD29, CD117, CD34, CD31 and/or CD11b/c.

3. The method according to 2, wherein the BM-MNCs contain CD34-positive cells at a percentage of 1.5% or higher.

4. A composition for prevention or treatment of neurologic function deficits in a vertebrate with spinal cord injury, which comprises BM-MNCs containing CD34-positive cells at a percentage of 1.5% or higher.
